(19) United States
(12) Patent Application Publication
(10) Pub. No.: US 2013/0158523 A1
(43) Pub. Date: Jun. 20, 2013

(54) Medical Devices, Apparatuses, Systems, and Methods for Magnetic Transmural and/or Transdermal Activation of Medical Tools

(71) Applicants: Richard A. Bergs, Grand Prarie, TX (US); Heather E. Beardsley, Arlington, TX (US); Jeffrey A. Cadeddu, Dallas, TX (US); Raul Fernandez, Arlington, TX (US); Kevin M. Huey, Cincinnati, OH (US); Daniel J. Scott, Dallas, TX (US)

(72) Inventors: Richard A. Bergs, Grand Prarie, TX (US); Heather E. Beardsley, Arlington, TX (US); Jeffrey A. Cadeddu, Dallas, TX (US); Raul Fernandez, Arlington, TX (US); Kevin M. Huey, Cincinnati, OH (US); Daniel J. Scott, Dallas, TX (US)

(21) Appl. No.: 13/331,426

(22) Filed: Dec. 20, 2011

(51) Int. Cl. A61B 17/00

(52) U.S. Cl. 606/1

(57) ABSTRACT

Systems, methods, apparatuses, and medical devices configured for transmural and/or transdermal magnetic actuation of a tool of a medical device (e.g., without translation of the medical device and/or relative to a platform of the medical device).

### BACKGROUND

1. Field of the Invention

The present invention relates generally to medical devices, apparatuses, systems, and methods, and, more particularly, but not by way of limitation, to medical devices, apparatuses, systems, and methods for performing medical procedures at least partially within a body cavity of a patient.

2. Description of Related Art

For illustration, the background is described with respect to medical procedures (e.g., surgical procedures), which can include laparoscopy, transmural surgery, and endoluminal surgery, including, for example, natural orifice transluminal endoscopic surgery (NOTES), single-incision laparoscopic surgery (SILS), and single-port laparoscopy (SLP).

Compared with open surgery, laparoscopy can result in significantly less pain, faster convalescence and less morbidity. NOTES, which can be an even less-invasive surgical approach, may achieve similar results. However, issues such as eye-hand dissociation, a two-dimensional field-of-view, instrumentation with limited degrees of freedom, and demanding dexterity requirements can pose challenges for many laparoscopic and endoscopic procedures. One limitation of laparoscopy can be the fixed working envelope surrounding each trocar. As a result, multiple ports may be used to accommodate changes in position of the instruments or laparoscope, for example, to improve visibility and efficiency. However, the placement of additional working ports may contribute to post-operative pain and increases risks, such as additional bleeding and adjacent organ damage.

The following published patent applications include information that may be useful in understanding the present medical devices, systems, and methods, and each is incorporated by reference in its entirety: (1) International Application No. PCT/US2009/063987, filed on Nov. 11, 2009, and published as WO 2010/056716; (2) U.S. patent application Ser. No. 10/024,636, filed Dec. 14, 2001, and published as Pub. No. US 2003/0114731; (3) U.S. patent application Ser. No. 10/999,396, filed Nov. 30, 2004, published as Pub. No. US 2005/0165449, and issued as U.S. Pat. No. 7,429,259; (4) U.S. patent application Ser. No. 11/741,731, filed Apr. 28, 2007, published as Pub. No. US 2007/0255273 and issued as U.S. Pat. No. 7,691,103; (5) U.S. patent application Ser. No. 12/146,953, filed Jun. 26, 2008, and published as Pub. No. US 2008/0269779; (6) International Patent Application No. PCT/US10/21292, filed Jan. 16, 2010, and published as WO 2010/083480.

### SUMMARY

This disclosure includes embodiments of medical devices, apparatuses, systems, and methods.

Embodiments of the present medical devices comprise: a platform; a first element comprising at least one of a magnetically-attractive material and a magnetically-chargeable material coupled to the platform; and a second element comprising at least one of a magnetically-attractive material and a magnetically-chargeable material coupled to the platform; and a tool coupled to the platform; where the tool is configured to be moved substantially without translating the platform by moving an apparatus that is magnetically coupled to the second element but not in physical contact with the tool.

In some embodiments, the tool is configured to be moved relative to the platform. In some embodiments, the tool is configured to pivot relative to the platform around a pivot axis. In some embodiments, the first element is coupled in substantially fixed relation to the tool. In some embodiments, the pivot axis extends through the first element, and the first element is configured to pivot around the pivot axis. In some embodiments, the first element is magnetized along an axis that is not parallel to the pivot axis. In some embodiments, the first element has a substantially circular cross-sectional shape. In some embodiments, the first element is movably coupled to the platform, and the medical device further comprises: a link coupled to the first element and the tool such that moving the first element in a first direction causes the tool to rotate in a first rotational direction and moving the first element in a second direction causes the tool to rotate in a second direction. In some embodiments, the link is pivotally coupled to the first element and the tool. In some embodiments, the first element and the second element are configured to be magnetically coupled to an apparatus such that a coupling force of at least 500 grams is generated between the apparatus and the first and second elements at a distance of 10 millimeters between them.

Some embodiments further comprise: a third element comprising at least one of a magnetically-attractive material and a magnetically-chargeable material coupled to the platform; where the second element and the third element are coupled in fixed relation to the platform; and the first element is movable relative to the platform. In some embodiments, the second element and the third element are configured to be magnetically coupled to an apparatus such that a coupling force of at least 500 grams is generated between the apparatus and the second and third elements at a distance of 10 millimeters between them.

Some embodiments of the present apparatuses comprise: a platform configured to be magnetically coupled to a medical device disposed within a body cavity of a patient through a tissue (e.g., where the platform comprises: a first element comprising at least one of a magnet and magnetically-chargeable material; and a second element comprising at least one of a magnet and magnetically-chargeable material; where the first element is movable relative to the second element to move a tool of the medical device without contacting the medical device). In some embodiments, the first element is movable relative to the second element to move the tool relative to a platform of the medical device. Some embodiments further comprise: an actuator configured to move the first element relative to the second element. In some embodiments, the actuator includes a lever arm coupled to the first element such that moving a portion of the lever arm in a first direction causes the first element to move relative to the second element. In some embodiments, the first element is configured to rotate relative to the second element. In some embodiments, the lever arm comprises a first end and a second end coupled to the first element, the lever arm is pivotally coupled to the platform around a pivot axis between the first end and the second end such that movement of the first end in a first direction causes the first element to rotate in a first rotational direction.

Some embodiments of the present apparatuses further comprise: a third element comprising at least one of a magnet and magnetically-chargeable material; where the first element is movable relative to the second element and the third element. In some embodiments, the second element is substantially fixed relative to the third element. In some embodiments, the first element is coupled to the platform such that the first element is rotatable around a longitudinal axis relative to the platform, the third element is coupled to the platform such that the third element is rotatable around a longitudinal axis relative to the platform; and at least one of the first and third elements can be rotated relative to the platform to cause the medical device to rotate around a longitudinal axis of the medical device. In some embodiments, the longitudinal axis of the first element is substantially parallel to the longitudinal axis of the third element.

Some embodiments of the present systems comprise: a medical device configured to be inserted within a body cavity of a patient (e.g., where the medical device comprises: a platform; a first element comprising at least one of a magnetically-attractive material and a magnetically-chargeable material coupled to the platform; and a second element comprising at least one of a magnetically-attractive material and a magnetically-chargeable material coupled to the platform; and a tool coupled to the platform; where the tool is configured to be moved without translating the platform by moving an apparatus that is magnetically coupled to the second element but not in physical contact with the tool); and a second platform configured to be magnetically coupled to the first platform through a tissue (e.g., where the second platform comprises: a first element comprising at least one of a magnet and magnetically-chargeable material; and a second element comprising at least one of a magnet and magnetically-chargeable material; where the first element is movable relative to the second element to move a tool of the medical device without contacting the medical device).

Some embodiments of the present methods comprise: magnetically coupling an element outside the body cavity of a patient to a tool of a platform disposed in the body cavity of the patient, the tool coupled to the platform; and moving the tool relative to the platform inside the body cavity by moving the element outside the body cavity.

Some embodiments of the present methods comprise: magnetically coupling an embodiment of the present apparatuses to an embodiment of the present medical devices such that the apparatus does not physically contact the medical device; and moving the first element of the apparatus to cause the tool of the medical device to move substantially without translating the platform of the medical device.

Any embodiment of any of the present medical devices, apparatuses, systems, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiment depicted in the figures.

FIG. 1 depicts a graphical representation of one of the present medical devices positioned within a body cavity of a patient and magnetically coupled to a positioning apparatus that is located outside the cavity.

FIG. 2 is an end view of the medical device and positioning apparatus shown in FIG. 1.

FIGS. 3A-3B depict a bottom view and a side cross-sectional view, respectively, respectively, of an embodiment of the positioning apparatus shown in FIG. 1.

FIG. 4 depict side cross-sectional view of one embodiment of the present medical devices shown magnetically coupled to magnetically attractive and/or magnetically-chargeable elements of a first embodiment of the present apparatuses.

FIG. 5 depicts a side cross-sectional view of a second embodiment of the present medical devices shown magnetically coupled to magnetically attractive and/or magnetically-chargeable elements of a first embodiment of the present apparatuses.

FIG. 6 depicts a perspective view of the first embodiment of the present positioning apparatuses configurable for use with the medical devices of FIGS. 4 and 5.

FIG. 7 depicts a side cross-sectional view of the second embodiment of the present medical devices shown magnetically coupled to magnetically attractive and/or magnetically-chargeable elements of a second embodiment of the present apparatuses.

FIG. 8 depicts a perspective view of the second embodiment of the present apparatuses configurable for use with the medical device of FIG. 7.

FIGS. 9A-9B depict side cross-sectional views of a third embodiment of the present medical devices shown magnetically coupled to magnetically attractive and/or magnetically-chargeable elements of a second embodiment of the present apparatuses.

FIG. 10 depicts a perspective view of a third embodiment of the present apparatuses.

FIG. 11 depicts a perspective view of a fourth embodiment of the present medical devices.

FIG. 12 depicts an end cross-sectional view of an embodiment of the present systems including the positioning apparatus of FIG. 10 and the medical device of FIG. 11.

### DESCRIPTION OF ILLUSTRATIVE EMBODIMENTS

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a device or kit that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, a device or system that is configured in a certain way is configured in at least that way, but it can also be configured in other ways than those specifically described.

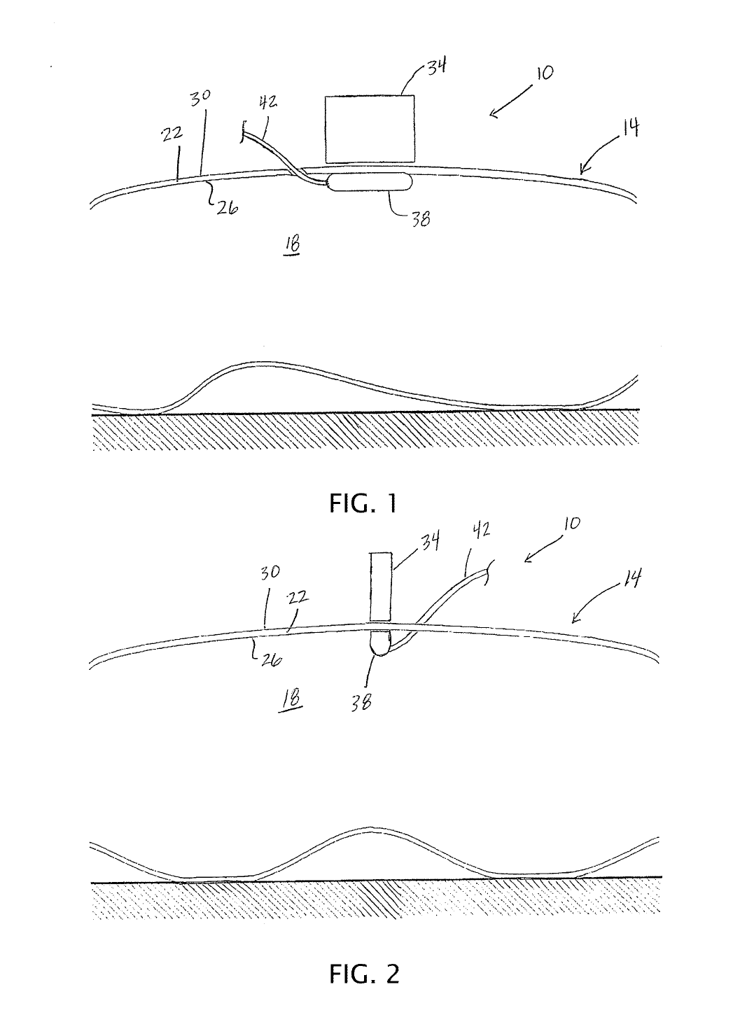

Referring now to the drawings, shown in FIGS. 1 and 2 by reference numeral 10 is one embodiment of a system for medical procedures that can be used with the present invention. System 10 is shown in conjunction with a patient 14, and more particularly in FIG. 1 is shown relative to a longitudinal cross-sectional view of the ventral cavity 18 of a human patient 14, and in FIG. 2 is shown relative to a transverse cross-sectional view of the ventral cavity of the patient. For brevity, cavity 18 is shown in simplified conceptual form without organs and the like. Cavity 18 is at least partially defined by wall 22, such as the abdominal wall, that includes an interior surface 26 and an exterior surface 30. The exterior surface 30 of wall 22 can also be an exterior surface 30 of the patient 14. Although patient 14 is shown as human in FIGS. 1 and 2, various embodiments of the present invention (including the version of system 10 shown in FIGS. 1 and 2) can also be used with other animals, such as in veterinary medical procedures.

Further, although system 10 is depicted relative to ventral cavity 18, system 10 and various other embodiments of the present invention can be utilized in other body cavities of a patient, human or animal, such as, for example, the thoracic cavity, the abdominopelvic cavity, the abdominal cavity, the pelvic cavity, and other cavities (e.g., lumens of organs such as the stomach, colon, or bladder of a patient). In some embodiments of the present methods, and when using embodiments of the present devices and systems, a pneumoperitoneum may be created in the cavity of interest to yield a relatively-open space within the cavity.

As shown in FIGS. 1 and 2, system 10 comprises an apparatus 34 and a medical device 38; the apparatus is configured to magnetically position the device with a body cavity of a patient. In some embodiments, apparatus 34 can be described as an exterior apparatus and/or external unit and device 38 as an interior device and/or internal unit due the locations of their intended uses relative to patients. As shown, apparatus 34 can be positioned outside the cavity 18 near, adjacent to, and/or in contact with the exterior surface 30 of the patent 14. Device 38 is positionable (can be positioned), and is shown positioned, within the cavity 18 of the patient 14 and near, adjacent to, and/or in contact with the interior surface 26 of wall 22. Device 38 can be inserted or introduced into the cavity 18 in any suitable fashion. For example, the device 18 can be inserted into the cavity through a puncture (not shown) in wall 22, through a tube or trocar (not shown) extending into the cavity 18 through a puncture or natural orifice (not shown), or may be inserted into another portion of the patient 14 and moved into the cavity 18 with apparatus 34, such as by the methods described in this disclosure. If the cavity 18 is pressurized, device 38 can be inserted or introduced into the cavity 18 before or after the cavity 18 is pressurized.

Additionally, some embodiments of system 10 include a version of device 38 that has a tether 42 coupled to and extending away from the device 38. In the depicted embodiment, tether 42 extends from device 38 and out of the cavity 18, for example, through the opening (not shown) through which device 38 is introduced into the cavity 18. The tether 42 can be flexible and/or elongated. In some embodiments, the tether 42 can include one or more conduits for fluids that can be used, for example, for actuating a hydraulic cylinder or irrigating a region within the cavity 18. In some embodiments, the tether 42 can include one or more conductors for enabling electrical communication with the device 38. In some embodiments, the tether 42 can include one or more conduits for fluid and one or more conductors. In some embodiments, the tether does not include a conduit or conductor and, instead, includes a cord for positioning, moving, or removing device 38 from the cavity 18. Tether 14, for example, can be used to assist in positioning the device 34 while the device 34 is magnetically coupled to the apparatus 38, or to remove the device 34 from the cavity 18 when device 38 is not magnetically coupled to apparatus 34. In other embodiments, the tether is omitted such that device 38 is controlled wirelessly from outside the body cavity.

As is discussed in more detail below, apparatus 34 and device 38 can be configured to be magnetically couplable to one another such that device 38 can be positioned or moved within the cavity 18 by positioning or moving apparatus 34 outside the cavity 18. “Magnetically couplable” means capable of magnetically interacting so as to achieve a physical result without a direct physical connection. Examples of physical results are causing device 38 to move within the cavity 18 by moving apparatus 34 outside the cavity 18, and causing device 38 to remain in a position within the cavity 18 or in contact with the interior surface 26 of wall 22 by holding apparatus 34 in a corresponding position outside the cavity 18 or in contact with the exterior surface 30 of wall 22. Magnetic coupling can be achieved by configuring apparatus 34 and device 38 to cause a sufficient magnetic attractive force between them. For example, apparatus 34 can comprise one or more magnets (e.g., permanent magnets, electromagnets, or the like) and device 38 can comprise a ferromagnetic material. In some embodiments, apparatus 34 can comprise one or more magnets, and device 38 can comprise a ferromagnetic material, such that apparatus 34 attracts device 38 and device 38 is attracted to apparatus 34. In other embodiments, both apparatus 34 and device 38 can comprise one or more magnets such that apparatus 34 and device 38 attract each other.

The configuration of apparatus 34 and device 38 to cause a sufficient magnetic attractive force between them can be a configuration that results in a magnetic attractive force that is large or strong enough to compensate for a variety of other factors (such as the thickness of any tissue between them) or forces that may impede a desired physical result or desired function. For example, when apparatus 34 and device 38 are magnetically coupled as shown, with each contacting a respective surface 26 or 30 of wall 22, the magnetic force between them can compress wall 22 to some degree such that wall 22 exerts a spring or expansive force against apparatus 34 and device 38, and such that any movement of apparatus 34 and device 38 requires an adjacent portion of wall 22 to be similarly compressed. Apparatus 34 and device 38 can be configured to overcome such an impeding force to the movement of device 38 with apparatus 34. Another force that the magnetic attractive force between the two may have to overcome is any friction that exists between either and the surface, if any, that it contacts during a procedure (such as apparatus 34 contacting a patient's skin). Another force that the magnetic attractive force between the two may have to overcome is the force associated with the weight and/or tension of the tether 42 and/or frictional forces on the tether 42 that may resist, impede, or affect movement or positioning of device 38 using apparatus 34.

In some embodiments, device 38 can be inserted into cavity 18 through an access port having a suitable internal diameter. Such access ports includes those created using a conventional laparoscopic trocar, gel ports, those created by incision (e.g., abdominal incision), and natural orifices. Device 38 can be pushed through the access port with any elongated instrument such as, for example, a surgical instrument such as a laparoscopic grasper or a flexible endoscope.

In embodiments where the tether 42 is connectable to a power source or a hydraulic source (not shown), the tether can be connected to the power source or the hydraulic source (which may also be described as a fluid source) either before or after it is connected to device 38.

In some embodiments, when device 38 is disposed within cavity 18, device 38 can be magnetically coupled to apparatus 34. This can serve several purposes including, for example, to permit a user to move device 38 within cavity 18 by moving apparatus 34 outside cavity 18. The magnetic coupling between the two can be affected by a number of factors, including the distance between them. For example, the magnetic attractive force between device 38 and apparatus 34 increases as the distance between them decreases. As a result, in some embodiments, the magnetic coupling can be facilitated by temporarily compressing the tissue (e.g., the abdominal wall) separating them. For example, after device 38 has been inserted into cavity 18, a user (such as a surgeon) can push down on apparatus 34 (and wall 22) and into cavity 18 until apparatus 34 and device 38 magnetically couple.

In FIGS. 1 and 2, apparatus 34 and device 38 are shown at a coupling distance from one another and magnetically coupled to one another such that device 38 can be moved within the cavity 18 by moving apparatus 34 outside the outside wall 22. The “coupling distance” between two structures (e.g., apparatus 34 and device 38) is defined as a distance between the closest portions of the structures at which the magnetic attractive force between them is great enough to permit them to function as desired for a given application.

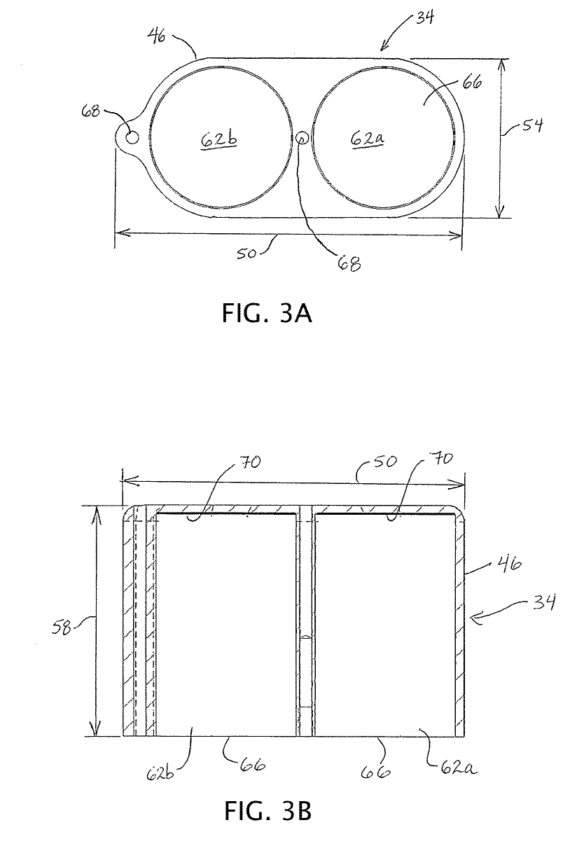

Referring now to FIGS. 3A and 3B, a bottom view and a side cross-sectional view are shown, respectively, of an embodiment of apparatus 34. Apparatus 34 has a width 50, a depth 54, and a height 58, and includes a housing 46. The apparatus (and, more specifically, housing 46) is configured to support, directly or indirectly, at least one magnetic assembly in the form of one or more magnetic field sources. In the embodiments shown, apparatus 34 is shown as including a first magnetic field source 62a and a second magnetic field source 62b. Each magnetic field source 62a, 62b has a coupling end 66 and a distal end 70. As described in more detail below, the coupling ends face device 38 when apparatus 34 and device 38 are magnetically coupled. The depicted embodiment of housing 46 of apparatus 34 also includes a pair of guide holes 68 extending through housing 46 for guiding, holding, or supporting various other devices or apparatuses, as described in more detail below. In other embodiments, the housing of apparatus 34 can have any other suitable number of guide holes 68 such as, for example, zero, one, three, four, five, or more guide holes 68. In some embodiments, housing 46 comprises a material that is minimally reactive to a magnetic field such as, for example, plastic, polymer, fiberglass, or the like. In other embodiments, housing 46 can be omitted or can be integral with the magnetic field sources such that the apparatus is, itself, a magnetic assembly comprising a magnetic field source.

Magnets, in general, have a north pole (the N pole) and a south pole (the S pole). In some embodiments, apparatus 34 can be configured (and, more specifically, its magnetic field sources can be configured) such that the coupling end 66 of each magnetic field source is the N pole and the distal end 70 of each magnetic field source is the S pole. In other embodiments, the magnetic field sources can be configured such that the coupling end 66 of each magnetic field source is the S pole and the distal end 70 of each magnetic field source is the N pole. In other embodiments, the magnetic field sources can be configured such that the coupling end of the first magnetic field source 62a is the N pole and the recessed end of the first magnetic field source 62a is the S pole, and the coupling end of the second magnetic field source 62b is the S pole and the recessed end of the second magnetic field source 62b is the N pole. In other embodiments, the magnetic field sources can be configured such that the coupling end of the first magnetic field source 62a is the S pole and its recessed end is the N pole, and the coupling end of the second magnetic field source 62b is the N pole and its recessed end is the S pole.

In the embodiment shown, each magnetic field source includes a solid cylindrical magnet having a circular cross section. In other embodiments, each magnetic field source can have any suitable cross-sectional shape such as, for example, rectangular, square, triangular, fanciful, or the like. In some embodiments, each magnetic field source comprises any of: any suitable number of magnets such as, for example, one, two, three, four, five, six, seven, eight, nine, ten, or more magnets; any suitable number of electromagnets such as, for example, one, two, three, four, five, six, seven, eight, nine, ten or more electromagnets; any suitable number of pieces of ferromagnetic material such as, for example, one, two, three, four, five, six, seven, eight, nine, ten or more pieces of ferromagnetic material; any suitable number of pieces of paramagnetic material such as, for example, one, two, three, four, five, six, seven, eight, nine, ten or more pieces of paramagnetic material; or any suitable combination of magnets, electromagnets, pieces of ferromagnetic material, and/or pieces of paramagnetic material. In some embodiments, each magnetic field source can include four cylindrical magnets (not shown) positioned in end-to-end in linear relation to one another, with each magnet having a height of about 0.5 inch and a circular cross-section that has a diameter of about 1 inch. In these embodiments, the magnets can be arranged such that the N pole of each magnet faces the S pole of the next adjacent magnet such that the magnets are attracted to one another and not repulsed.

Examples of suitable magnets can include: flexible magnets; Ferrite, such as can comprise Barium or Strontium; AlNiCo, such as can comprise Aluminum, Nickel, and Cobalt; SmCo, such as can comprise Samarium and Cobalt and may be referred to as rare-earth magnets; and NdFeB, such as can comprise Neodymium, Iron, and Boron. In some embodiments, it can be desirable to use magnets of a specified grade, for example, grade 40, grade 50, or the like. Such suitable magnets are currently available from a number of suppliers, for example, Magnet Sales & Manufacturing Inc., 11248 Playa Court, Culver City, Calif. 90230 USA; Amazing Magnets, 3943 Irvine Blvd. #92, Irvine, Calif. 92602; and K & J Magnetics Inc., 2110 Ashton Dr. Suite 1A, Jamison, Pa. 18929. In some embodiments, one or more magnetic field sources can comprise ferrous materials (e.g., steel) and/or paramagnetic materials (e.g., aluminum, manganese, platinum).

In some embodiments, apparatus 34 and device 38 can be configured to have a minimum magnetic attractive force or “coupling force” at a certain distance. For example, in some embodiments, apparatus 34 and device 38 can be configured such that at a distance of 50 millimeters between the closest portions of apparatus 34 and device 38, the magnetic attractive force between apparatus 34 and device 38 is at least about: 20 grams, 25 grams, 30 grams, 35 grams, 40 grams, or 45 grams. In some embodiments, apparatus 34 and device 38 can be configured such that at a distance of about 30 millimeters between the closest portions of apparatus 34 and device 38, the magnetic attractive force between them is at least about: 25 grams, 30 grams, 35 grams, 40 grams, 45 grams, 50 grams, 55 grams, 60 grams, 65 grams, 70 grams, 80 grams, 90 grams, 100 grams, 120 grams, 140 grams, 160 grams, 180 grams, or 200 grams. In some embodiments, apparatus 34 and device 38 can be configured such that at a distance of about 15 millimeters between the closest portions of apparatus 34 and device 38, the magnetic attractive force between them is at least about: 200 grams, 250 grams, 300 grams, 350 grams, 400 grams, 45 grams, 500 grams, 550 grams, 600 grams, 650 grams, 700 grams, 800 grams, 900 grams, or 1000 grams. In some embodiments, apparatus 34 and device 38 can be configured such that at a distance of about 10 millimeters between the closest portions of apparatus 34 and device 38, the magnetic attractive force between them is at least about: 500 grams, 1000 grams, 2000 grams, 2200 grams, 2400 grams, 2600 grams, 2800 grams, 3000 grams, 3200 grams, 3400 grams, 3600 grams, 3800 grams, or 4000 grams.

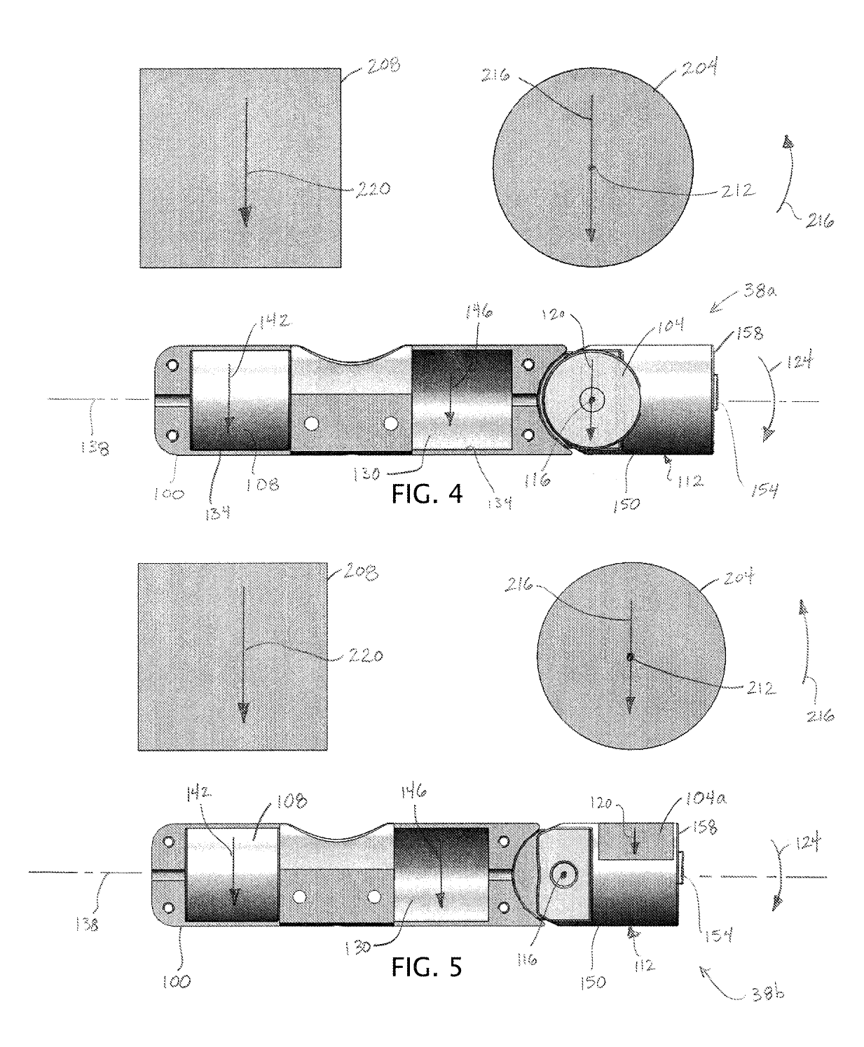

FIG. 4 depicts a side cross-sectional view of one embodiment 38a of the present medical devices. In the embodiment shown, device 38a comprises a platform 100; a first element 104 coupled to platform 100; a second element 108 coupled to platform 100; and a tool 112 coupled to platform 100. In the embodiment shown, first element 104 comprises at least one of a magnetically-attractive material and a magnetically-chargeable material, and second element 108 comprises at least one of a magnetically-attractive material and a magnetically-chargeable material. For example, first element 104 and second element 108 can each comprise a ferromagnetic material. In the embodiment shown, tool 112 is configured to be moved substantially without translating the body by moving an apparatus (e.g., 34) that is magnetically coupled to the second element but not in physical contact with the tool. For example, and as illustrated, a first element 204 of a control apparatus (e.g., 34) can be magnetically coupled (e.g., through a patient's tissue) to first element 104 of device 34a, and a second element 208 of a control apparatus can be magnetically coupled to second element 108 of device 34a.

In the embodiment shown, first element 204 of the apparatus can be configured to rotate relative to second element 208 to move tool 112 relative to platform 100. For example, in the embodiment shown, first element 204 is provided with a circular cross-sectional shape, and is configured to rotate around a pivot axis 212. In the embodiment shown, first element 204 is diametrically magnetized and/or magnetizable (e.g., in direction 216) such that rotation of first element 204 changes direction 216 of magnetization relative to device 38a. In the embodiment shown, second element 208 has a rectangular cross-sectional shape and is magnetized and/or magnetizable in a direction 220. In other embodiments, second element 208 can have any suitable shape and/or can be magnetized and/or magnetizable in any suitable direction.

In the embodiment shown, first element 104 of device 38a is coupled in substantially fixed relation to tool 212, and is configured to pivot around a pivot axis 116 (and such that pivot axis 116 extends through first element 104). In the embodiment shown, first element 104 has a substantially circular cross-sectional shape (in a cross-sectional plane that is substantially perpendicular to pivot axis 116), and is diametrically magnetized and/or magnetizable in direction 120. Thus, if first element 104 of device 34a is magnetically coupled to first element 204, rotation of first element 204 in a counter-clockwise direction 216 will cause rotation of first element 104 (and tool 112) of device 38a in a clockwise direction 124. In other embodiments, first element 104 can have any suitable cross-sectional shape and/or can be magnetized and/or magnetizable in any suitable direction that is not parallel to pivot axis 116 (e.g., can be disposed at an angle of 30, 45, 60, 75, or more degrees relative to pivot axis 116).

In some embodiments, device 38a can be configured such that first element 104 and second element 108 are configured to be magnetically coupled to an apparatus (e.g., 34, such as, for example, to a first element 204 and a second element 208 of such an apparatus) such that a coupling force of at least 500 grams is generated between the apparatus and first and second elements 104 and 108 at a distance of 10 millimeters between them. For example, in some embodiments, device 38a includes only first element 104 and second element 108, such that they are substantially the only elements of device 38a that directly contribute to magnetic coupling with the apparatus.

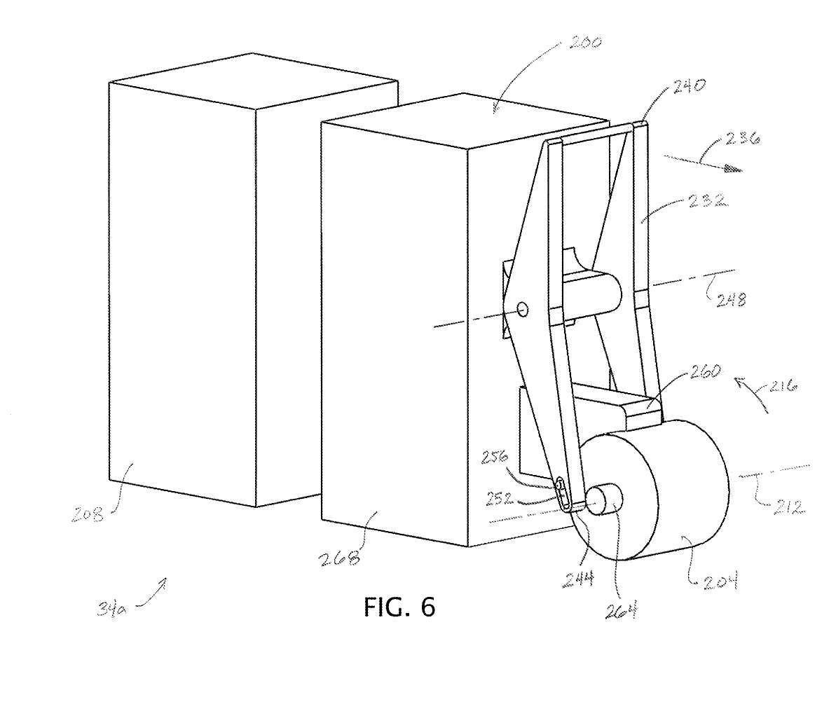

In the embodiment shown, device 38a, further comprises: a third element 130 comprising at least one of a magnetically-attractive material and a magnetically-chargeable material coupled to platform 100. In the embodiment shown, second element 108 and third element 130 are coupled in fixed relation to platform 100, and first element 104 is movable relative to the platform. In the embodiment shown, third element 130 can be configured to correspond to a third element of an apparatus (e.g., 34, which is not shown in FIG. 4, but one example of which is depicted, for example, in FIG. 6). In the embodiment shown, second element 108 and third element 130 are configured to be coupled to an apparatus (e.g., 34, such as, for example, to a corresponding second element 208 and a third element of such an apparatus) such that a coupling force of at least 500 grams is generated between the apparatus and the second and third elements at a distance of 10 millimeters between them. In such embodiments, for example, the coupling force generated between the apparatus and the second and third elements can be sufficient for positioning platform 100, such that first element 104 need not contribute to the overall coupling force.

Second and third elements 108 and 130 can comprise any suitable material that is magnetically attracted to the magnetic field sources 62a and 62b of apparatus 34 (and/or first and second elements 208 and 268 of apparatus 34a, described below). Examples of such material include, for example, a magnet, a ferromagnetic material, and a paramagnetic material. In some embodiments of the present devices, e.g., device 38a, each of second and third elements 108 and 130 comprises a cylindrical magnet. In other embodiments, each of second and third elements 108 and 130 comprises a plurality of magnets (e.g., of varying sizes or shapes) such as, for example, five cylindrical magnets having a circular cross-section. In other embodiments, second and third elements 108 and 130 have or include any suitable cross-sectional shape, dimension, or number of magnets, or volumes of ferromagnetic or paramagnetic materials. In embodiments of the present devices, e.g. device 38a, where second and third elements 108 and 130 include magnets, each of the second and third elements will generally have an N pole and an S pole. In some of these embodiments, the second and third elements are magnetized in opposite directions (e.g., in an N-S/S-N configuration or S-N/N-S configuration).

In the embodiment shown, platform 100 includes interior openings 134 configured to receive second element 108 and third element 130. In the embodiment shown, each of second element 108 and third element 130 has a circular cross-sectional shape (in a cross-sectional plane that is perpendicular to a longitudinal axis 138 of platform 100), and are diametrically magnetized and/or magnetizable in directions 142 and 146, respectively. In other embodiments, second element 108 and/or third element 130 can have any suitable shape and/or be magnetized and/or magnetizable in any suitable direction.

In the embodiment shown, tool 112 comprises a housing 150 and a camera 154 having a field-of-view FOV extending outward from a distal end 158 of housing 150. In this embodiment, rotation of tool 112 as described adjusts the angle of the FOV of the camera such that rotation of first element 204 (if magnetically coupled to first element 104) can change the angle of tool 112 (and FOV of camera 154). In other embodiments, tool 112 can comprise any suitable configuration or components (e.g., scalpel, cautery, hook, and/or the like). In some embodiments, tool 112 (e.g., housing 150) can be biased (e.g., via a spring or other resilient member disposed around axis 116) toward a neutral position, such as that shown in FIG. 4 in which housing 150 is substantially aligned with and parallel to platform 100.

FIG. 5 depicts a side cross-sectional view of a second embodiment 38b of the present medical devices. Device 38b is substantially similar in many respects to device 38a, and similar reference numerals are used to denote elements of device 38b that are similar to elements of device 38a. Likewise, in the embodiment shown, first element 204 and second element 208 are substantially similar to those described in FIG. 4, with the exception that the distance between first element 204 and 208 is larger in FIG. 5. As such, the differences between device 38a and device 38b are primarily described here. In the embodiment shown, device 38b includes first element 104a that is coupled in fixed relation to tool 212 and housing 150 and is spaced apart from pivot axis 116, as shown (such that pivot axis 116 does not extend through first element 104a), such that rotation of first element 204 in a counter-clockwise direction 216 will reduce the coupling force between first element 204 and first element 104a of device 38b to permit tool 112 (and camera 154) to pivot downward in clockwise direction 124. In the embodiment shown, first element 104a also has a rectangular cross-sectional shape (in a plane perpendicular to rotational axis 116).

FIG. 6 depicts a perspective view of one embodiment 34a of the present positioning apparatuses that can be configured for use with the medical devices of FIGS. 4 and 5. Apparatus 38a is substantially similar in some respects to apparatus 38 depicted in FIGS. 3A and 3B, and includes elements (e.g., first and second elements 204 and 208) depicted and described with reference to FIGS. 4 and 5. As such, the differences between apparatus 38a and 38 are primarily described here. In the embodiment shown, apparatus 34 comprises a platform 200 that is configured to be magnetically coupled to a medical device (e.g., 38a, 38b) disposed within a body cavity of a patient through a tissue. In the embodiment shown, platform 200 comprises: a first element 204 comprising at least one of a magnet and magnetically-chargeable material; and a second element 208 comprising at least one of a magnet and magnetically-chargeable material. In the embodiment shown, first element 204 is movable relative to second element 208 to move a tool (e.g., 212) of the medical device without contacting the medical device.

In the embodiment shown, first element 204 is movable relative to second element 208 to move the tool (e.g., 212) relative to a platform (e.g., 100) of the medical device (e.g., 38a, 38b). For example, the apparatus can comprise an actuator 228 configured to move first element 204 relative to second element 208. In the embodiment shown, actuator 228 includes a lever arm 232 coupled to first element 204 such that moving a portion of lever arm 232 in a first direction 236 causes the first element to move (e.g., to rotate, as shown) relative to the second element. For example, in the embodiment shown, lever arm 232 comprises a first end 240 and a second end 244 coupled to first element 208, and the lever arm is pivotally coupled to platform 200 around a pivot axis 248 between first end 240 the second end 244 such that movement of first end 240 in direction 236 causes first element 208 to rotate in a first rotational direction 216. In the embodiment shown, second end 244 is slidably and pivotally coupled to first element 204 via a slot 252 into which a pin 256 extends. Thus, in the embodiment shown, when first end 240 is moved in direction 236, second end 248 moves in a direction opposing direction 236 to cause first element 204 to rotate in rotational direction 216. In the embodiment shown, member 260 extends outward from platform 200 to maintain rotational axis 212 in substantially fixed relation to platform 200. Member 260 can be coupled to first element in any suitable manner or with any suitable structure, such as, for example, magnetically coupled, a portion of member 260 extending through first element 204 (e.g., through a radial slot or the like in first member 204), a fork extending from member 260 to axle 264 of first element 204, and/or any other coupling manner or structure that permits apparatus 34a to function as described.

In some embodiments, apparatus 34a can be configured such that first element 204 and second element 208 are configured to be magnetically coupled to a medical device (e.g., 38a, 38b, such as, for example, to a first element 104 and a second element 108 of such an apparatus) such that a coupling force of at least 500 grams is generated between the medical device and first and second elements 204 and 208 at a distance of 10 millimeters between them. For example, in some embodiments, apparatus 34a includes only first element 204 and second element 208, such that they are substantially the only elements of apparatus 34a that directly contribute to magnetic coupling with the medical device.

In the embodiment shown, apparatus 34a also comprises a third element 268 comprising at least one of a magnet and magnetically-chargeable material, and first element 204 is movable relative to platform 200 (including second element 208 and third element 268). In the embodiment shown, third element 268 is substantially fixed relative to second element 208. In the embodiment shown, third element 268 is configured to a correspond to a third element (e.g., 130) of a medical device (e.g., 38a, 38b). In the embodiment shown, second element 208 and third element 268 are configured to be coupled to a medical device (e.g., 38a, 38b) such that a coupling force of at least 500 grams is generated between the medical device and the second and third elements at a distance of 10 millimeters between them. In such embodiments, for example, the coupling force generated between the medical device and the second and third elements of apparatus 34a can be sufficient for positioning the medical device, such that first element 204 need not contribute to the overall coupling force. In some embodiments, second element 208 and third element 268 are substantially similar to first magnetic field source 62a and a second magnetic field source 62b, as described above and depicted in FIGS. 3A and 3B. In some embodiments, third element 268 is magnetized and/or magnetizable in a direction that is opposite direction 216.

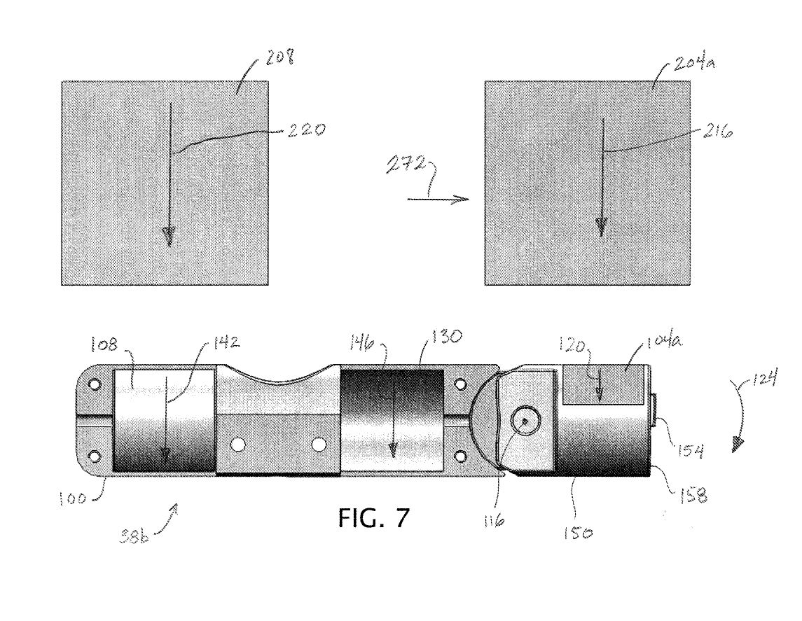

FIG. 7 depicts a side cross-sectional view of the second embodiment 38b of the present medical devices shown magnetically coupled to magnetically attractive and/or magnetically-chargeable elements 204a and 208 of a second embodiment 34b of the present apparatuses. First and second elements 204a and 208 are substantially similar in some respects to first element 204 and second element 208, and similar reference numerals are used to denote features of first and second elements 204a and 208 that are similar to first and second elements 204 and 208. As such, the differences between first element 204a and first element 204 are primarily described here. In the embodiment shown, first element 204a has a substantially rectangular shape, and is magnetized and/or magnetizable in a direction 216. In the embodiment shown, first element 204a is configured to translate (instead of rotate) relative to second element 208, such that the orientation of direction 216 remains substantially constant (does not pivot). In this embodiment, translation of first element 204a in direction 272 can increase the distance between first element 204a of apparatus 34b and first element 104a of device 38b to decrease the coupling force therebetween and permit tool 112 (camera 154) to pivot downward in direction 124. Similarly, translating first element 204a back in a direction opposite to direction 272 has the opposite effect and encourages tool 112 to pivot in a direction opposite to direction 124.

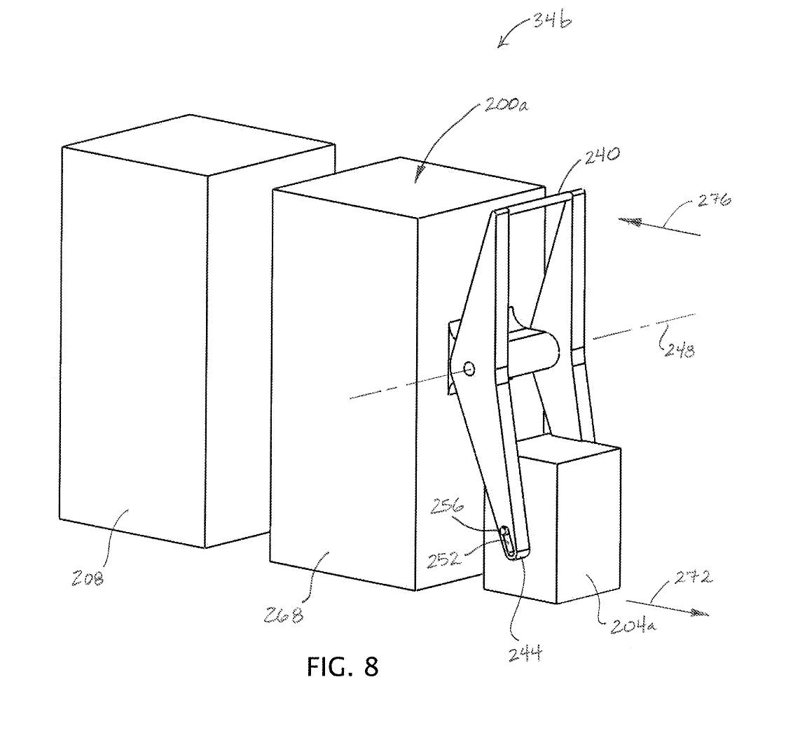

FIG. 8 depicts a perspective view of a second embodiment 34b of the present positioning apparatuses that is configured for use with device 38b of FIG. 7. Apparatus 34b is substantially similar in some respects to apparatus 34a, and similar numerals are used to denote elements of apparatus 34b that are similar to elements of apparatus 34a. As such, the differences between apparatus 34b and apparatus 34a are primarily described here. In the embodiment shown, apparatus 34b comprises first element 204a that is configured to move laterally relative to platform 200a without pivoting (such that the orientation of direction 216 remains substantially constant). The orientation of first member 204a can be maintained relative to platform 200a in any suitable manner or with any suitable structure, such as, for example, magnetically coupled, a guide or rail extending outward from platform 200a adjacent to and/or through first element 204a (e.g., through slot or the like in first member 204a, and/or any other coupling manner or structure that permits apparatus 34a to function as described. Thus, in the embodiment shown, apparatus 34b is configured such that movement of first end 240 in direction 276 will cause second end 244 and first element 204a to move in opposite direction 272 (and thus cause tool 112 of device 38b to rotate downward in direction 124).

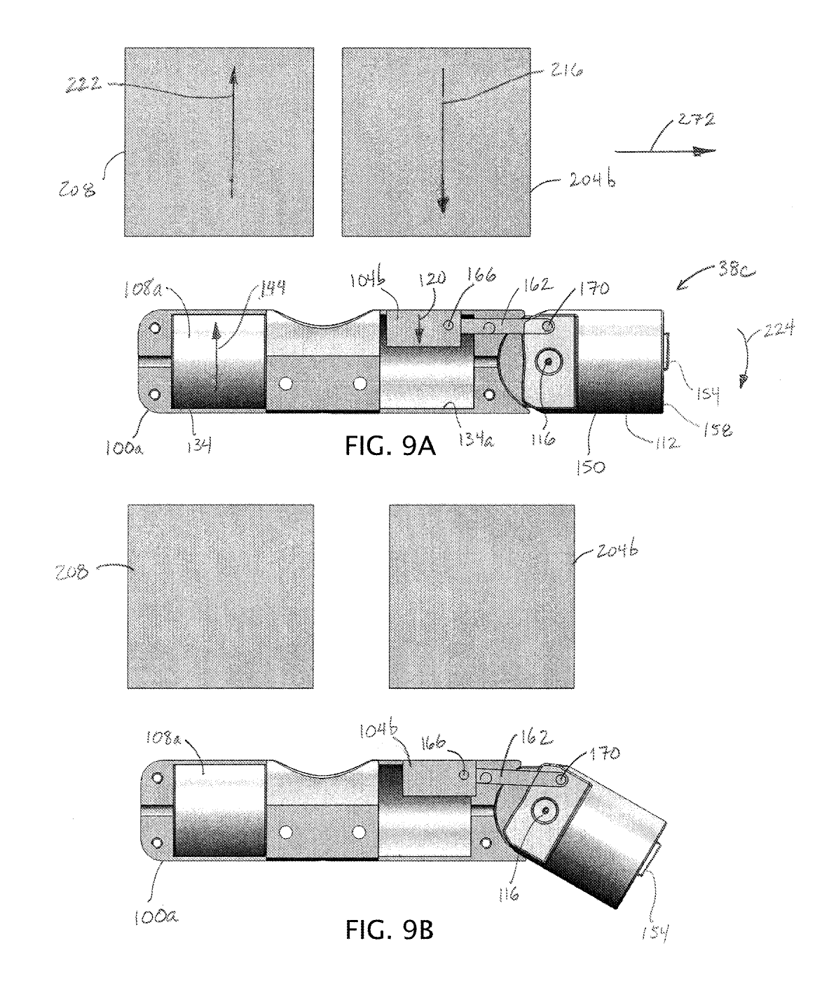

FIGS. 9A and 9B depict side cross-sectional views of a third embodiment 38c of the present medical devices shown magnetically coupled to magnetically attractive and/or magnetically-chargeable elements 204b and 208. First element 204b is substantially similar to third element 268, with the primary difference that first element 204b is movable relative to second element 208. Device 38c is substantially similar in some respects to devices 38b, and similar reference numerals are used to denote elements of device 38c that are similar to elements of device 38b. As such, the differences between device 38c and device 38a are primarily described here. In the embodiment shown, first element 104b is movably coupled to platform 100a such that first element 104b is spaced apart from pivot axis 116 (and such that pivot axis does not extend through first element 104b).

In some embodiments, first element 104b can be configured to translate relative to the platform, and coupled to the tool such that translation of the first member results in rotation of the tool. For example, in the embodiment shown, device 38c also comprises a link 162 coupled to first element 104b and to tool 112 such that moving first element 104b in a first direction 272 causes the tool to rotate in direction 124 as shown in FIG. 9B, and moving first element 104b in a second direction opposite to direction 272 causes tool 112 to rotate in a second direction opposite direction 124. More particularly, in this embodiment, link 162 is pivotally coupled to first element 104b via a pin or axle 166 and to the tool (e.g., housing 150) via a pin or axle 170. In the embodiment shown, platform 100a is configured to slidably receive first element 104b in opening 134a. In this embodiment, device 38c does not include a third magnetically-attracting and/or magnetically-chargeable element, and instead, first element 104b and second element 108 are configured to be coupled to produce the desired coupling force when magnetically coupled to an apparatus (e.g., 38). As such, an apparatus (e.g., 38) can be configured such that the two magnetic field sources (e.g., 62a, 62b) or elements (e.g., 204b, 208) can be configured to be movable laterally relative to one another to actuate the tool. For example, in the embodiment shown, if first element 204b is magnetically coupled to first element 104b and second element 208 is magnetically coupled to second element 108, then first element 204b can be moved in direction 272 to cause first element 104b to also move in direction 272, and thereby cause tool 112 to rotate in direction 124. In the embodiment shown, second element 208 is magnetized and/or magnetizable in a direction 222 that is opposite direction 216, and second element 108a of device 38c is magnetized and/or magnetizable in direction 144 that is substantially opposite direction 120. Second element 108a is similar in other respects to second element 108, described above. In some embodiments, tool 112 (e.g., housing 150) can be biased (e.g., via a spring or other resilient member disposed between first element 104b and platform 100a) toward a neutral position, such as that shown in FIG. 9A in which housing 150 is substantially aligned with and parallel to platform 100a.

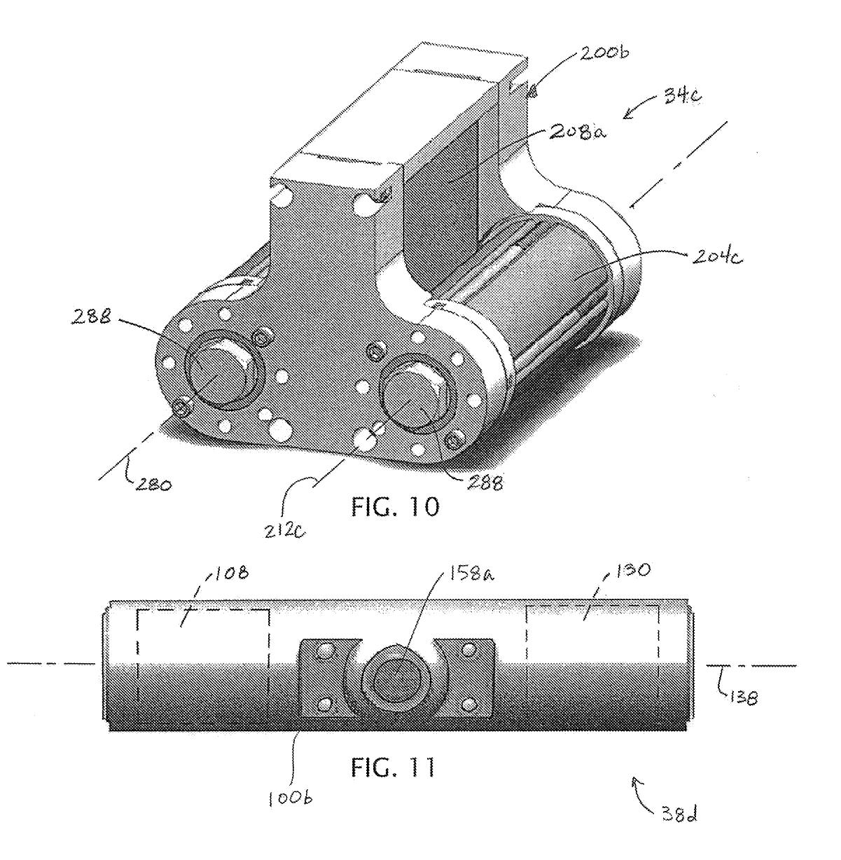

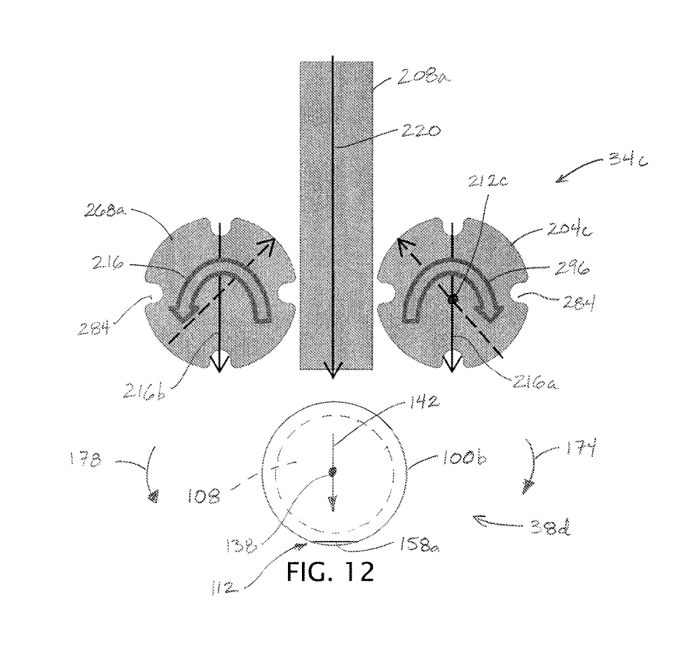

Referring now to FIGS. 10-12, FIG. 10 depicts a perspective view of a third embodiment 34c of the present positioning apparatuses, FIG. 11 depicts a perspective view of a fourth embodiment 38d of the present medical devices that can be used with apparatus 34c, and FIG. 12 depicts an cross-sectional view of an embodiment of the present systems including apparatus 34c and device 38d taken at the longitudinal center of the apparatus and the device. Apparatus 34c is substantially similar in some respects to apparatus 34b, and similar numerals are used to denote elements of apparatus 34c that are similar to elements of apparatus 34b. As such, the differences between apparatus 34c and apparatus 34b are primarily described here. Likewise, device 38d is substantially similar in some respects to device 38b, and similar numerals are used to denote elements of device 38d that are similar to elements of device 38b. As such, the differences between device 38d and device 38b are primarily described here.

In the embodiment shown, apparatus 34c comprises a platform 200b, a first element 204c, and a second element 208a. In the embodiment shown, each of first and second elements 204c and 208a comprising at least one of a magnetically-attractive material and a magnetically-chargeable material coupled to the platform. In the embodiment shown, first element 204c is coupled to the platform such that the first element is rotatable around a longitudinal axis 212a relative to the platform and second element 208a. In the embodiment shown, apparatus 34c further comprises a third element 268a is coupled to the platform such that the third element is rotatable around a longitudinal axis 280 relative to the platform. In the embodiment shown, second element 208a has a rectangular cross-sectional shape (in a plane perpendicular to axes 212a and 280), and is magnetized and/or magnetizable in a direction 220a.

In the embodiment shown, each of first element 204c and third element 268a comprises a substantially circular cross-sectional shape (in a plane perpendicular to axes 212a and 280, which, in the embodiment shown, are substantially parallel) with recessed portions 284 configured to receive rods 288 (e.g., bolts) that may, in some embodiments, be configured to maintain the relative orientations of components of the respective first or second element. In some embodiments, each of first and third elements can comprise a single component or piece of material, and/or recessed portions 284 can be omitted. In the embodiment shown, each of first and third elements 204c and 268a is diametrically magnetized and/or magnetizable in a respective direction 216a or 216b. In the embodiment shown, each of first and third elements 204c and 268a is pivotally coupled to platform 100b by a bolt or other axle 292. In such embodiments, each of first and third elements 204c and 268a is coupled in fixed relation to the respective bolt 292 such that rotation of the bolt (e.g., via a wrench, socket, wingnut, protrusion, or any other suitable structure coupled to or extending from the bolt) causes rotation of the respective first or third element. In the embodiment shown, the first and third elements are configured to be rotated in independently and/or in the same rotational direction (e.g., both clockwise or both counterclockwise).

In the embodiment shown, device 38d comprises a platform 100b, a second element 108, and a third element 130. In the embodiment shown, device 38d comprises a tool 112 in the form of a camera 154a that is in fixed relation to platform 100b (at the center of the platform, in this embodiment). Thus, movement of the camera and its FOV depends on movement of the entire platform 100b. In the embodiment shown, apparatus 34c is configured to move the camera by causing device 38d to rotate around its longitudinal axis 138. In particular, in the embodiment shown, apparatus 34c is configured such that at least one (e.g., both) of first and third elements 204c and 268a can be rotated relative to platform 200b to cause device 38d to rotate around longitudinal axis 138. For example, in the embodiment shown, first element 204c and/or third element 268a can be rotated in direction 296 to cause device 38d to rotate in direction 174. Likewise, in the embodiment shown, first element 204c and/or third element 268a can be rotated in direction 216 to cause device 38d to rotate in direction 178.

Embodiments of the present systems include an apparatus (e.g., 34, 34a, 34b, 34c, 34d) configured to be magnetically coupled (e.g., magnetically coupled) to a medical device (e.g., 38, 38a, 38b, 38c, 38d).

Embodiments of the present methods can comprise: magnetically coupling an element (e.g., 204, 204a, 204b, 204c) outside the body cavity of a patient to a tool (e.g., 212, such as, for example, via an element 104, 104a, 104b) of a platform (e.g., 100, 100a, 100b) disposed in the body cavity of the patient, where the tool is coupled to the platform; and moving the tool relative to the platform inside the body cavity by moving the element outside the body cavity. Some embodiments of the present methods comprise: magnetically coupling an embodiment of the present apparatuses (e.g., 34a, 34b, 34c, 34d) to an embodiment of the present medical devices (e.g., 38a, 38b, 38c, 38d) such that the apparatus does not physically contact the medical device; and moving the first element of the apparatus to cause the tool of the medical device to move substantially without translating the platform of the medical device.

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present devices are not intended to be limited to the particular forms disclosed. Rather, they include all modifications and alternatives falling within the scope of the claims, and embodiments other than the one shown may include some or all of the features of the depicted embodiment. For example, components may be combined as a unitary structure, and/or connections may be substituted (e.g., threads may be substituted with press-fittings or welds). Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A medical device comprising:
a platform;
a first element comprising at least one of a magnetically-attractive material and a magnetically-chargeable material coupled to the platform; and
a second element comprising at least one of a magnetically-attractive material and a magnetically-chargeable material coupled to the platform; and
a tool coupled to the platform;
where the tool is configured to be moved substantially without translating the platform by moving an apparatus that is magnetically coupled to the second element but not in physical contact with the tool.

2. The medical device of claim 1, where the tool is configured to be moved relative to the platform.

3. The medical device of claim 2, where the tool is configured to pivot relative to the platform around a pivot axis.

4. The medical device of claim 3, where the first element is coupled in substantially fixed relation to the tool.

5. The medical device of claim 4, where the pivot axis extends through the first element, and the first element is configured to pivot around the pivot axis.

6. The medical device of claim 5, where the first element is magnetized along an axis that is not parallel to the pivot axis.

7. The medical device of claim 6, where the first element has a substantially circular cross-sectional shape.

8. The medical device of claim 3, where the first element is movably coupled to the platform, and the medical device further comprises:
a link coupled to the first element and the tool such that moving the first element in a first direction causes the tool to rotate in a first rotational direction and moving the first element in a second direction causes the tool to rotate in a second direction.

9. The medical device of claim 8, where the link is pivotally coupled to the first element and the tool.

10. The medical device of claim 1, where the first element and the second element are configured to be magnetically coupled to an apparatus such that a coupling force of at least 500 grams is generated between the apparatus and the first and second elements at a distance of 10 millimeters between them.

11. The medical device of claim 1, further comprising:
a third element comprising at least one of a magnetically-attractive material and a magnetically-chargeable material coupled to the platform;
where the second element and the third element are coupled in fixed relation to the platform; and the first element is movable relative to the platform.

12. The medical device of claim 11, where the second element and the third element are configured to be magnetically coupled to an apparatus such that a coupling force of at least 500 grams is generated between the apparatus and the second and third elements at a distance of 10 millimeters between them.

13. An apparatus comprising:
a platform configured to be magnetically coupled to a medical device disposed within a body cavity of a patient through a tissue, the platform comprising:
a first element comprising at least one of a magnet and magnetically-chargeable material; and
a second element comprising at least one of a magnet and magnetically-chargeable material;
where the first element is movable relative to the second element to move a tool of the medical device without contacting the medical device.

14. The apparatus of claim 13, where the first element is movable relative to the second element to move the tool relative to a platform of the medical device.

15. The apparatus of claim 13, further comprising:
an actuator configured to move the first element relative to the second element.

16. The apparatus of claim 16, where the actuator includes a lever arm coupled to the first element such that moving a portion of the lever arm in a first direction causes the first element to move relative to the second element.

17. The apparatus of claim 16, where the first element is configured to rotate relative to the second element.

18. The apparatus of claim 17, where the lever arm comprises a first end and a second end coupled to the first element, the lever arm is pivotally coupled to the platform around a pivot axis between the first end and the second end such that movement of the first end in a first direction causes the first element to rotate in a first rotational direction.

19. The apparatus of claim 13, further comprising:
a third element comprising at least one of a magnet and magnetically-chargeable material;
where the first element is movable relative to the second element and the third element.

20. The apparatus of claim 19, where the second element is substantially fixed relative to the third element.

21. The apparatus of claim 19, where the first element is coupled to the platform such that the first element is rotatable around a longitudinal axis relative to the platform, the third element is coupled to the platform such that the third element is rotatable around a longitudinal axis relative to the platform; and at least one of the first and third elements can be rotated relative to the platform to cause the medical device to rotate around a longitudinal axis of the medical device.

22. The apparatus of claim 21, where the longitudinal axis of the first element is substantially parallel to the longitudinal axis of the third element.

23. A system comprising:
a medical device configured to be inserted within a body cavity of a patient, the medical device comprising:
a platform;
a first element comprising at least one of a magnetically-attractive material and a magnetically-chargeable material coupled to the platform; and
a second element comprising at least one of a magnetically-attractive material and a magnetically-chargeable material coupled to the platform; and
a tool coupled to the platform;
where the tool is configured to be moved without translating the platform by moving an apparatus that is magnetically coupled to the second element but not in physical contact with the tool; and
a second platform configured to be magnetically coupled to the first platform through a tissue, the second platform comprising:
a first element comprising at least one of a magnet and magnetically-chargeable material; and
a second element comprising at least one of a magnet and magnetically-chargeable material;
where the first element is movable relative to the second element to move a tool of the medical device without contacting the medical device.

24. A method comprising:
magnetically coupling an element outside the body cavity of a patient to a tool of a platform disposed in the body cavity of the patient, the tool coupled to the platform;
moving the tool relative to the platform inside the body cavity by moving the element outside the body cavity.

25. A method comprising:
magnetically coupling an apparatus of claim 13 to a medical device of claim 1 such that the apparatus does not physically contact the medical device; and
moving the first element of the apparatus to cause the tool of the medical device to move substantially without translating the platform of the medical device.
